(19) United States
(12) Patent Application Publication
(10) Pub. No.: US 2017/0311811 A1
(43) Pub. Date: Nov. 2, 2017

(54) INFORMATION ACQUISITION APPARATUS

(71) Applicant: CANON KABUSHIKI KAISHA, Tokyo (JP)

(72) Inventor: Yukio Furukawa, Sagamihara-shi (JP)

(73) Assignee: CANON KABUSHIKI KAISHA, Tokyo (JP)

(21) Appl. No.: 15/526,290

(22) PCT Filed: Oct. 22, 2015
(86) PCT No.: PCT/JP2015/005324

§ 371 Date: May 11, 2017

(30) Foreign Application Priority Data
Nov. 18, 2014 (JP) 2014-233799

(51) Int. Cl. A61B 5/00; F21V 8/00

(52) U.S. Cl. CPC A61B 5/0095; G02B 6/0008; A61B 5/4312; A61B 5/748

(57) ABSTRACT

To suppress variation of a distribution of radiated light depending on a position of a radiating unit in an information acquisition apparatus that uses an articulated arm as a waveguide unit. A waveguide unit (103) includes a plurality of first waveguides (103c), (103g), and (103k) that guide light in a direction parallel to a radiating direction in which the light is radiated from a radiating unit (105) to an object (123), at least one of second waveguides (103a), (103e), and (103i) that guides the light in an in-plane direction perpendicular to the radiating direction, and a plurality of articulations that each include therein a mirror disposed so as to substantially perpendicularly bend a wave guiding direction. The light is guided through the plurality of first waveguides (103c), (103g), and (103k) in the same wave guiding direction.

### TECHNICAL FIELD

The present invention relates to an information acquisition apparatus.

### BACKGROUND ART

Optical imaging apparatuses are actively studied in the medical field. The optical imaging apparatuses image information in an object to be tested. The information in the object is acquired in accordance with light radiated from a light source such as a laser to the object and incident upon the object. One of examples of such an optical imaging technology is the photo acoustic tomography (referred to as PAT hereafter).

With the PAT, pulsed light emitted from a light source is radiated to the object. The light propagates through and is scattered in the object is absorbed by tissue, and consequently an acoustic wave is generated. This acoustic wave is detected and a received signal is acquired. The PAT is a technology that acquires information about optical characteristics inside a living body as the object by analyzing the received signal. This phenomenon of generation of a photoacoustic wave is referred to as a photoacoustic effect, and an acoustic wave generated by the photoacoustic effect is referred to as a photoacoustic wave. With the above-described technology, an optical characteristic distribution, in particular an optical absorption coefficient distribution inside the object can be acquired. The above-described information can also be utilized for quantitative measurement of a specific substance inside the object, for example, glucose or hemoglobin contained in blood.

it is known that the strength of the photoacoustic wave or a received signal of the photoacoustic wave is proportional to the optical absorption coefficient of the source of the photoacoustic wave and the energy density of light radiated to the source. That is, when imaging the optical absorption coefficient distribution inside the object, accurate acquisition of a distribution of the light energy density (referred to as light intensity distribution hereafter) inside the object is effective for improvement of quantitativity of the optical absorption coefficient distribution.

Since arrangement of the light source near the object is physically restricted, the light source unit is typically connected to the radiating unit, which radiates pulsed light to the object, through a waveguide unit.

PTL 1 discloses an example in which a light source and an emitting unit (radiating unit) are connected through an articulated arm (waveguide unit) in a laser treatment apparatus. This articulated arm includes a plurality of rigid pipes that allow light to propagate through hollows of the rigid pipes and a plurality of articulations that include mirrors therein. The radiating position can be changed by the articulated arm. Furthermore, according to PTL 1, information of the emitting unit such as a laser transmittance and a radiation spot diameter is stored in advance, and the conditions of the radiated light are optimized with reference to the information of the installed light emitting unit.

### CITATION LIST

### Patent Literature

PTL 1: Japanese Patent Laid-Open No. 2003-613

### SUMMARY OF INVENTION

### Technical Problem

With a waveguide unit that uses an articulated arm, a radiated light intensity distribution of light radiated by a radiating unit may vary depending on a radiating position of the radiating unit. When such variation of the radiated light intensity distribution is not considered, the light intensity distribution in an object cannot be accurately acquired. Consequently, a distribution of the optical absorption coefficient cannot be accurately acquired.

The present invention suppresses variation of a radiated light intensity distribution depending on a scanning position of a radiating unit in an information acquisition apparatus that uses an articulated arm as a waveguide unit.

### Solution to Problem

An information acquisition apparatus according to the present invention includes a light source, a radiating unit, a waveguide unit, a detection unit, and an acquisition unit. The radiating unit radiates to an object light emitted from the light source. The waveguide unit guides the light emitted from the light source to the radiating unit. The detection unit detects an acoustic wave generated by radiating the light from the radiating unit to the object and outputs an electrical signal. The acquisition unit acquires information of an inside of the object in accordance with the electric signal. The radiating unit is able to perform scanning and connected to an end point of the waveguide unit. The waveguide unit includes a plurality of first waveguides, at least one second waveglide, and a plurality of articulations. The plurality of first waveguides guide the light in a direction parallel to a radiating direction in which the light is radiated from the radiating unit to the object. The at least one second waveguide guides the light in an in-plane direction perpendicular to the radiating direction. The plurality of articulations connect the plurality of first waveguides and the at least one second waveguide to one another and each include therein a mirror disposed so as to substantially perpendicularly bend a wave guiding direction of the light guided through the plurality of first waveguides and the at least one second waveguide. The plurality of first waveguides are connected to one side and another side of the at least one second waveguide with the plurality of articulations interposed therebetween. The wave guiding direction of the light guided through one of the plurality of first waveguides located on the one side of the at least one second waveguide is identical to the wave guiding direction of the light guided through the other of the plurality of first waveguides located on the other side of the at least one second waveguide.

### Advantageous Effects of Invention

According to the present invention, variation of a radiated light intensity distribution depending on a position of a radiating unit can be suppressed in an information acquisition apparatus that uses an articulated arm as a waveguide unit.

Further features of the present invention will become apparent from the following description of exemplary embodiments with reference to the attached drawings.

### BRIEF DESCRIPTION OF DRAWINGS

FIG. 1A is a schematic view of an example of an information acquisition apparatus according to a first embodiment the present invention.

FIG. 1B is a schematic view of the example of the information acquisition apparatus according to the first embodiment of the present invention.

FIG. 2A illustrates a state of a light intensity distribution in a waveguide unit according to a comparative example.

FIG. 2B illustrates the state of the light intensity distribution in the waveguide unit according to the comparative example.

FIG. 2C illustrates the state of the light intensity distribution in the waveguide unit according to the comparative example.

FIG. 3A illustrates a state of a light intensity distribution in a waveguide unit according to the first embodiment of the present invention.

FIG. 3B illustrates the state of the light intensity distribution in the waveguide unit according to the first embodiment of the present invention.

FIG. 3C illustrates the state of the light intensity distribution in the waveguide unit according to the first embodiment of the present invention.

FIG. 4 illustrates a scanning path of a radiating unit according to the first embodiment of the present invention.

FIG. 5 is a schematic view of an example of an information acquisition apparatus according to a second embodiment.

FIG. 6 illustrates a scanning path and a region of interest where information is acquired in an information acquisition apparatus according to the second embodiment of the present invention.

FIG. 7 is a schematic view of an example of an information acquisition apparatus according to a third embodiment of the present invention.

FIG. 8 is a schematic view of an example of a waveguide unit used in an information acquisition apparatus according to a fourth embodiment of the present invention.

### DESCRIPTION OF EMBODIMENTS

An information acquisition apparatus according to the present invention will be described below. In the present invention, an acoustic wave refers to an elastic wave generated in an object by radiating light such as near infrared light (electromagnetic wave) to the object. Examples of the elastic wave include waves referred to as a sonic wave, an ultrasonic wave, and a photoacoustic wave. The information acquisition apparatus according to the present invention acquires object information of the inside of the object mainly for diagnosis of malignant tumors, vascular diseases, and the like of human and animal, a follow-up observation of chemotherapy, and so forth. Accordingly, the object is considered to be a living body, specifically a human body or animal body, and a target area for diagnosis is considered to be part of the living body such as, for example, a breast, a finger, or a limb.

The object information according to the present embodiment includes information such as a light energy absorption density, an optical absorption coefficient, concentrations of substances included in tissue, and a sound pressure (initial sound pressure) of a photoacoustic wave generated due to a photo-acoustic effect. Here, examples of the concentrations of substances include an oxygen saturation, a concentration of oxyhemoglobin, a concentration of deoxyhemoglobin, a total concentration of the hemoglobin, and so forth. The total concentration of the hemoglobin refers to the sum of the concentration of oxyhemoglobin and the concentration of deoxyhemoglobin.

The object information according to the present embodiment is not necessarily numeric data. The object information may be distribution data. That is, the object information may be the distribution data such as an optical absorption coefficient distribution and an oxygen saturation distribution. The object information may be in the form of image data.

The information acquisition apparatus according to the present invention includes a light source, a radiating unit, and a waveguide unit. The radiating unit radiates light emitted from the light source to the object. The waveguide unit guides the light emitted from the light source to the radiating unit. The information acquisition apparatus also includes a detection unit and an acquisition unit. The detection unit detects the acoustic wave generated by radiating the light from the radiating unit to the object and outputs electrical signals. The acquisition unit acquires information of the inside of the object in accordance with the electrical signals.

### Light Source

When the object is a living body, the light source generates a pulsed light tuned to a wavelength at which the light is absorbed by a specific component out of components of the living body. In order to efficiently generate the photoacoustic wave, the pulse width can be from about 10 to 100 ns. The light source can be a laser with which a large output can be acquired. Alternatively, another light source such as a light emitting diode or a flash lamp may be used. Examples of the laser that can be used include various types of lasers such as a solid-state laser, a gas laser, a dye laser, and a semiconductor laser. The wavelength of the light source used in the present invention can be a wavelength at which the light propagates into the inside of the object. Specifically, the wavelength is 500 to 1200 nm in the case where the object is the living body.

A single light source may be used or a plurality of light sources may be used. In the case where the plurality of light sources are used, the plurality of light sources may generate light of a single spectrum band or different spectrum bands. The light source may be a tunable light source the central wavelength of which is variable.

### Radiating Unit

The radiating unit radiates the pulsed light emitted from the light source to the object such as the living body. The radiating unit can be adjusted by using optical elements such as a mirror, a lens, and a prism so that radiation strength, a light intensity distribution, and the position on the object are desirable. The radiating unit can perform one-dimensional scanning or two-dimensional scanning, and positions to be radiated by the radiating unit can be changed.

### Waveguide Unit

The waveguide unit guides the wave of the light emitted from the light source to the radiating unit. The waveguide unit includes an optical system in which a plurality of hollow waveguides are connected by articulations that include mirrors therein. The waveguide unit is connected to the light source and also connected to the radiating unit. Furthermore, some of the waveguides included in the waveguide unit are movable. The detailed structure will be described later.

### Detection Unit

The detection unit receives the photoacoustic wave generated on the surface of the object and in the inside of the object by the radiated pulsed light and converts the photoacoustic wave into electric signals (received signals) which is analogue signals. The detection unit may use detectors of any type that utilizes, for example, piezoelectric phenomena, resonance of light, or changes in electrostatic capacitance as long as the detector can receive acoustic wave signals. The detection unit can typically include a plurality of receiving elements disposed in a one-dimensional, two-dimensional, or three-dimensional arrangement. With the elements arranged in a multi-dimension, the acoustic wave can be simultaneously detected at a plurality of positions, and accordingly, measurement time can be reduced. When detectors are disposed in a threedimensional arrangement, the detectors can be arranged such that directional ranges of the detectors in which the receiver sensitivities of the detectors are strong are superposed on one another at the position of the object. For example, the detectors can be arranged along a spherical surface.

### Acquisition Unit

The acquisition unit acquires the object information of the inside of the object in accordance with the electric signals collected by an electric signal collection unit, which will be described later, and information about an emitted-light-intensity distribution corresponding to a single scanning position of the radiating unit stored in a memory, which will be described later. Specifically, the acquisition unit generates a threedimensional initial sound pressure distribution in the object from the electric signals collected by the electric signal collection unit. Regarding the generation of the initial sound pressure distribution, for example, a universal back-projection (UBP hereafter) algorithm or delay-and-sum algorithm may be used. Furthermore, the acquisition unit generates three-dimensional light intensity distribution information in the object in accordance with the stored information about the emitted-light-intensity distribution of the radiating unit. This information can be acquired by solving a light diffusion equation in accordance with information about the two-dimensional emitted-light-intensity distribution. The optical absorption coefficient distribution in the object as the object information can be acquired by normalizing the initial sound pressure distribution in the object generated from the electric signals by using the three-dimensional light intensity distribution information generated from the emitted-light-intensity distribution of the radiating unit, Furthermore, by computing the optical absorption coefficient distribution at a plurality of wavelengths, the oxygen saturation distribution of hemoglobin in the object can be acquired.

The information acquisition apparatus includes the following elements other than the above described elements.

### The Electric Signal Collection Unit

The electric signal collection unit collects the electric signals acquired by the detection unit. The electric signal collection unit can include an AID converter that converts analogue signals into digital signals for efficient processing.

### Holding Member

A holding member is used to hold the object and includes, for example, a cup-shaped structure following the shape of the object or two holding plates that hold the object therebetween so as to secure the object. The holding member or the holding plates positioned between the object and the detectors can have a low light absorption property and a low acoustic wave absorption property. In addition, the difference in acoustic impedance between the object and the holding member or the holding plates can be small. Such a holding member or holding plates can be formed of, for example, polymethylpentene resin.

### Scanning Unit

The scanning unit allows the radiating unit to perform two-dimensional scanning. The scanning unit may be provided with a position detection unit that detects a scanning position of the radiating unit during the scanning. The scanning unit may allow the detection unit and the radiating unit integrated with the scanning unit to simultaneously perform the scanning according to need.

### Scanning Driver

A scanning driver controls the scanning unit in accordance with an instruction from a controller, which will be described later, so as to cause the radiating unit to perform desired scanning. The scanning driver may drive the scanning unit so that the scanning by the scanning unit is continuously performed at a constant speed or may drive the scanning unit to perform a movement and data reception by a step and repeat method. The scanning driver may drive the scanning unit so that the scanning unit scans along an arc-shaped path or a spiral path.

### Scanning Position Acquisition Unit

A scanning position acquisition unit acquires the scanning position of the radiating unit when the pulsed light is radiated to the object. In the case where the position of the radiating unit can be recognized in accordance with the instruction issued from the controller to the scanning driver, the position detection unit and the scanning position acquisition unit are not necessarily required. In the case where the detection unit and the radiating unit are integrated with each other, the scanning position acquisition unit can simultaneously acquire position information of the detection unit when the pulsed light is radiated to the object.

### Controller

The controller controls so that the acoustic wave can be detected at desired timing. The controller includes a light source controller, a scanning controller, an electric signal collection controller, and a system controller, which will be described later.

### Light Source controller

The light source controller controls light emitting timing of the pulsed light, that is, timing at which the pulsed light is radiated to the object. For example, the light source controller causes the pulsed light to be emitted at a specific repetition frequency or causes the pulsed light to be emitted with reference to position information of the radiating unit.

### Scanning Controller

The scanning controller controls the scanning driver so as to cause the radiating unit to be desirably moved. Furthermore, the scanning controller issues an instruction to the scanning position acquisition unit so as to cause the scanning position acquisition unit to acquire the position information of the radiating unit at the moment when the pulsed light is radiated to the object. In order to allow acoustic information to be acquired from a specific region of interest of the object, the scanning controller may have a separate function that allows an operator to specify the region of interest and may issue a scanning instruction corresponding to the region of interest to the scanning driver.

### Electric Signal Collection Controller

The electric signal collection controller controls timing and a period of time at and during which the detection unit detects the acoustic wave generated in the object. The electric signal collection controller issues an instruction to the electric signal collection unit so as to collect the electric signals from the moment when the pulsed light is radiated to the object or from when a certain period of time has passed from the moment when the pulsed light was radiated to the object for a period of time corresponding to the depth of the object which is wanted to be imaged.

### System Controller

In order to allow the acoustic wave to be detected at desired timing, the light source controller, the scanning controller, and the electric signal collection controller are controlled so that the light source controller, the scanning controller, and the electric signal collection controller cooperate with one another.

### Memory

The memory stores information about the emitted-light-intensity distribution of light radiated from the radiating unit represented by, for example, a two-dimensional spatial distribution. It is sufficient that information about the emitted-light-intensity distribution be information at time when the radiating unit is disposed at a specified position. The emitted-light-intensity distribution is a light intensity distribution of the emitted light when a virtual screen is placed near the radiating unit or a position separated from the radiating unit by a certain distance.

The structure of the information acquisition apparatus according to the present invention has been described. The structure will be described in more detail in the following embodiments.

### First Embodiment

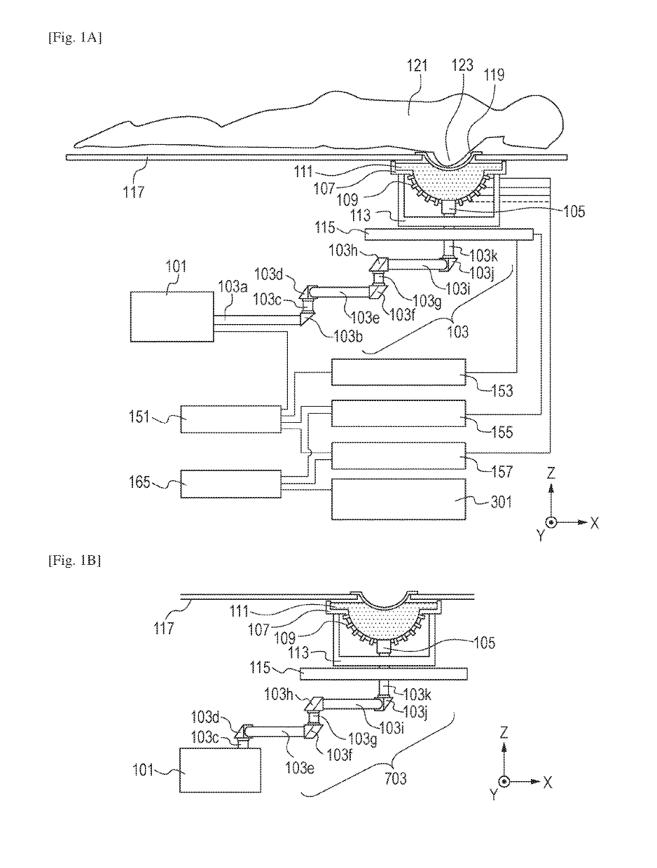

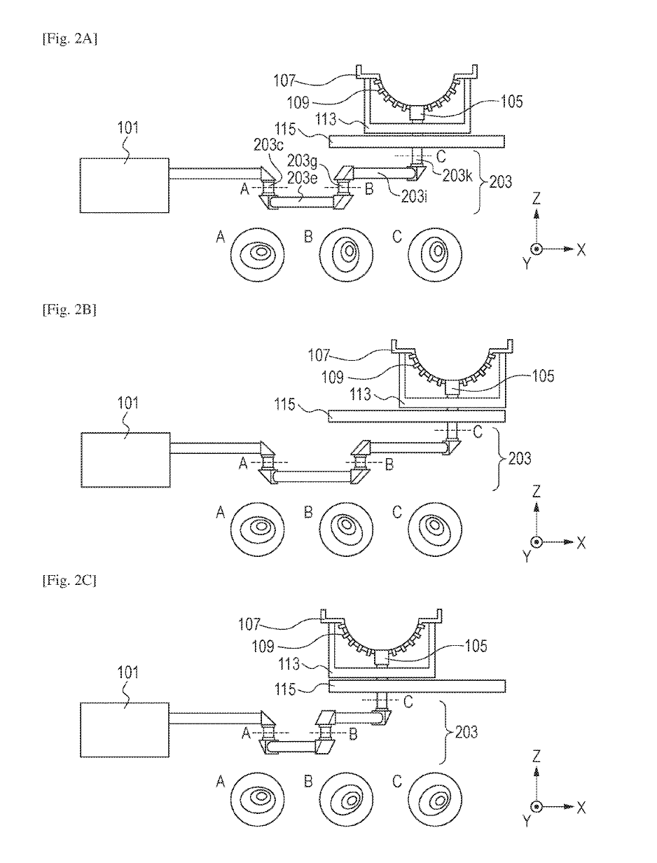

FIG. 1A is a schematic view illustrating an example of an information acquisition apparatus according to a first embodiment. The information acquisition apparatus includes a light source 101, a radiating unit 105, and a waveguide unit 103. The radiating unit 105 radiates light emitted from the light source 101 to the object (breast 123). The waveguide unit 103 guides the light emitted from the light source 101 to the radiating unit 105. The information acquisition apparatus also includes a detection unit 109 and an acquisition unit 165. The detection unit 109 detects an acoustic wave generated by radiating the light from the radiating unit 105 to the object and outputs electrical signals. The acquisition unit 165 acquires information of the inside of the object in accordance with the electrical signals. The radiating unit 105 can perform scanning.

The waveguide unit 103 includes an articulated arm. The articulated arm includes a plurality of first waveguides (corresponding to vertical waveguides 103c, 103g, and 103k of the present embodiment) and at least one second waveguide (corresponding to horizontal waveguides 103a, 103e, and 103i of the present embodiment). Furthermore, the articulated arm includes articulations (103b, 103d, 103f, 103h, and 103j). The vertical waveguides 103c, 103g, and 103k each have the function of guiding the light in a direction parallel to a radiating direction (+Z direction) in which the light is radiated from the radiating unit 105 to the object. The horizontal waveguides 103a, 103e, and 103i each have the function of guiding the light in an in-plane direction (XY in-plane direction) perpendicular to the radiating direction (+Z direction). The articulations connect the vertical waveguides and the horizontal waveguides to one another and each include therein a mirror disposed so as to substantially perpendicularly bend a wave guiding direction of the light guided through the vertical waveguides and the horizontal waveguides. Here, “to substantially perpendicularly bend” means to bend at an angle from 85 to 95 degrees.

The vertical waveguide 103c is connected to one side of the horizontal waveguide 103e with the articulation 103d interposed therebetween, and the vertical waveguide 103g is connected to the other side of the horizontal waveguide 103e with the articulation 103f interposed therebetween. The vertical waveguide 103g is connected to one side of the horizontal waveguide 103i with the articulation 103h interposed therebetween, and the vertical waveguide 103k is connected to the other side of the horizontal waveguide 103i with the articulation 103j interposed therebetween.

The horizontal waveguide 103a, the articulation 103b, and the vertical waveguide 103c are connected to one another so as not to be movable and secured in respective XY planes. The articulation 103d, the horizontal waveguide 103e, and the articulation 103f are connected to one another so as not to be independently movable. The articulation 103h, the horizontal waveguide 103i, and the articulation 103j are also connected to one another so as not to be independently movable. The articulation 103d is rotatable in the XY plane about the central axis of the vertical waveguide 103c. The articulations 103f and 103h are rotatable in the respective XY planes about the central axis of the vertical waveguide 103g. The articulation 103j is rotatable in the XY plane about the central axis of the vertical waveguide 103k. With this structure, the horizontal waveguides 103e and 103i and the vertical waveguides 103g and 103k are movable in the respective XY planes.

The light is guided through the plurality of vertical waveguides 103c, 103g, and 103k in the same wave guiding direction. Specifically, the direction of the light guided through any of the vertical waveguides 103c, 103g, and 103k is in the +Z direction. It is noted that the +Z direction and the −Z direction are distinguished from each other according to the present invention. A technical meaning of this structure will be described below.

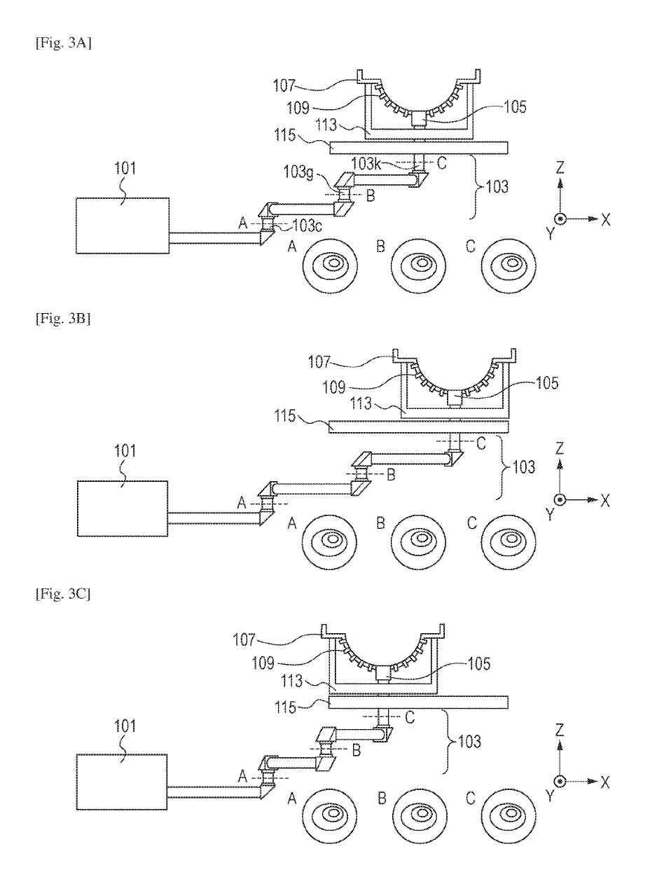

FIGS. 2A to 2C illustrate a waveguide unit 203 of an information acquisition apparatus of a comparative example. FIGS. 3A to 3C illustrate the waveguide unit 103 of the information acquisition apparatus according to the present embodiment. As described above, through all of the vertical waveguides 103c, 103g, and 103k, the light is guided in the same direction (+Z direction) according to the present embodiment. In contrast, in the comparative example, the light is guided through a vertical waveguide 203c in the −Z direction, and the light is guided through vertical waveguides 203g and 203k in the +Z direction. That is, in the waveguide unit 203 of the comparative example, the light is guided through in a different direction in one of the vertical waveguides 203c, 203g, and 203k from the other of the vertical waveguides 203c, 203g, and 203k. State of the emitted-light-intensity distribution of the radiated light due to this structural difference when the radiating unit 105 is moved is described below. The structure of the comparative example is the same as that of the present embodiment other than the above description.

The light source 101 is structured such that, in an optical path through which the light emitted from the light source 101 is guided through the waveguide unit 103, the emitted-light-intensity distribution of the light guided through the first waveguide (vertical waveguide 103c) closest to the light source 101 is asymmetric about the central axis of the vertical waveguide 103c.

The waveguide unit 203 that is an articulated arm of FIG. 2A is in a “bent elbow” state. The radiating unit 105 of FIG. 2B is moved rightward (+X direction) relative to the waveguide unit 203 of FIG. 2A. The waveguide unit 203 is in a “stretched elbow” state. However, the waveguide unit 203 of FIG. 2B is not in a “fully stretched elbow” state. FIG. 2C illustrates the case where the radiating unit 105 is moved leftward (−X direction). The waveguide unit 203 is in a “considerably bent elbow” state.

Referring to FIGS. 2A to 2C, screens (denoted by “A”, “B”, and “C” in the FIGS. 2A to 2C) are virtually placed in three vertical waveguides 203c, 203g, and 203k of the waveguide unit 203. Here, the case where emitted-light-intensity distributions of the light radiated to the screens are observed from a bed side is discussed, Examples of the emitted-light-intensity distributions are illustrated in circles of FIGS. 2A to 2C. The emitted-light-intensity distributions on the screens A and B are minor images of each other with the section of a horizontal waveguide 203e disposed therebetween set as the plane of symmetry. Accordingly, the emitted-light-intensity distribution on the screen A is reversed on the screen B. Furthermore, when observed from the bed side, the plane of symmetry (section of the horizontal waveguide 203e) is rotated in accordance with an “elbow angle” about the vertical waveguide 203c. As a result, the emitted-light-intensity distribution on the screen B is reversed and rotated compared to the emitted-light-intensity distribution on the screen A. In contrast, the emitted-light-intensity distribution on the screen B is maintained in the screen C and the orientation of the emitted-light-intensity distribution on the screen B is unchanged in the screen C even after the light has been guided through the horizontal waveguide 203i between the screens B and C.

From variations in the emitted-light-intensity distribution on the screens of FIGS. 2A to 2C, it can be understood that, as “the elbow is bent” further in the waveguide unit 203 (in the order of FIGS. 2A, 2B, and 2C), the emitted-light-intensity distribution on the screen C that is equal to the emitted-light-intensity distribution of the radiating unit 105 is rotated clockwise. Furthermore, the optical axis of the concave lens (not illustrated) of the radiating unit 105 may be misaligned with the center of a light beam having reached the radiating unit 105. In this case, by “stretching the elbow” of the waveguide unit 203, the radiated light from the radiating unit 105 is rotated about a position different from the center of the light beam. That is, with the structure of the waveguide unit 203 of the comparative example, the emitted-light-intensity distribution varies from scanning position to scanning position of the radiating unit 105. When the emitted-light-intensity distribution varies, a radiated light intensity distribution of the light which is actually radiated to the object varies. In this case, when using the emitted-light-intensity distribution of the radiating unit 105 corresponding to a certain radiating position without considering the variation in the emitted-light-intensity distribution, the acquisition unit 165 cannot accurately calculate the light intensity distribution in the object, As a result, the object information cannot be accurately acquired.

FIGS. 3A to 3C illustrate states of the emitted-light-intensity distributions in the vertical waveguides 103c, 103g, and 103k of the waveguide unit 103 according to the present embodiment. FIGS. 3A to 3C respectively correspond to FIGS. 2A to 2C.

From the emitted-light-intensity distributions on the screens of FIGS. 3A to 3C, it can be understood that, even when “the elbow is bent” in the waveguide unit 103, the emitted-light-intensity distribution on the screen C that is equal to the emitted-light-intensity distribution at the radiating unit 105 does not vary. Furthermore, when the optical axis of the concave lens (not denoted in FIGS. 3A to 3C) of the radiating unit 105 is misaligned with the center of the light beam having reached the radiating unit 105, the light radiated from the radiating unit 105 is inclined relative to the vertical direction. However, since the direction of the inclination is maintained independently of “bending and stretching of the elbow” of the waveguide unit 103, the emitted-light-intensity distribution does not vary. Accordingly, by using only the emitted-light-intensity distribution at the radiating unit 105 at a certain radiating position, the acquisition unit 165 can accurately calculate the light intensity distribution in the object. As a result, the object information can be accurately acquired.

The emitted-light-intensity distribution at the radiating unit 105 to be stored is information. acquired by measuring the emitted-light-intensity distribution of the light having been radiated when the radiating unit 105 is placed immediately below the bottom of a holding cup 119 that is at the center of a scannable range and the screen is disposed at a position above (+Z direction) the radiating unit 105 by 10 cm. This distance of 10 cm matches the radius of a semispherical support member that supports the detection unit 109, As described above, since the emitted-light-intensity distribution is substantially unchanged from scanning position to scanning position of the radiating unit 105 with the waveguide unit 103, the emitted-light-intensity distribution information to be stored in a memory 301 may be information acquired by measuring the emitted-light-intensity distribution at another scanning position in a similar manner. The information about the emitted-light-intensity distribution to be stored may instead be, for example, information acquired by measuring the emitted-light-intensity distribution of the light having been radiated when the radiating unit 105 is disposed at a position corresponding to the periphery of the holding cup 119 and the screen is disposed at a position above (+Z direction) the radiating unit 105 by 10 cm. Alternatively, the emitted-light-intensity distribution of the light having been radiated may be measured by disposing the screen at any position other than the position 10 cm above the radiating unit 105. The emitted-light-intensity distribution measured as described above is stored in the memory 301. That is, the memory 301 stores in advance the information about the emitted-light-intensity distribution of the emitted light radiated from the radiating unit 105 at a single scanning position out of a plurality of scanning positions scanned by the radiating unit 105. With this configuration, use of the storage capacity of the memory 301 can be reduced.

FIG. 1B is a schematic view of another example of the waveguide unit according to the present embodiment, This waveguide unit 703 introduces light from an upper portion of the light source 101 thereinto. The waveguide unit 703 does not include the horizontal waveguide 103a and the articulation 103b of the waveguide unit 103 of FIG. 1A. Furthermore, the vertical waveguide 103c is fixed. As is the case with the structure of FIG. 1A, the plurality of vertical waveguides 103c, 103g, and 103k guide the light therethrough in the same wave guiding direction (+Z direction).

When the radiating unit scans in a two-dimensional manner, the information acquisition apparatus needs two or more horizontal waveguides that is movable in the XY plane. Since the vertical waveguides and the horizontal waveguides are provided in an alternate sequence, two or more vertical waveguides serving as first waveguides movable in the XY plane are also needed. Furthermore, since an increase in the number of waveguides included in the waveguide unit 103 leads to a complex structure, the number of the vertical waveguides serving as the first waveguides is preferably five or less.

The light source 101 is, for example, a pulsed light source that uses a titanium-sapphire laser generating pulsed light of a wavelength of 800 nm, a pulse width of 20 ns, a repetition frequency of 10 Hz, and pulse energy of 30 mJ.

The radiating unit 105 includes the concave lens (not illustrated) therein that diverges the light beam. An end portion of the vertical waveguide 103k is connected to the radiating unit 105.

The detection unit 109 includes, for example, 500 transducers each include a piezoelectric element having a size of 3 mm square and a central detection frequency of 2 MHz. The transducers are arranged on a semispherical surface. The radius of the semisphere is 10 cm.

In addition to these elements, the information acquisition apparatus also includes a carriage (support body) 107 and a support table 113. The carriage 107 supports the radiating unit 105 and the detection unit 109 integrated with the radiating unit 105. The support table 113 supports the carriage 107, The support table 113 is provided on an XY stage (corresponding to the scanning unit) 115 and capable of two-dimensional scanning in the XY plane. The XY stage 115 is caused to perform scanning by a scanning driver 153. The XY stage 115 also includes a position sensor (not illustrated).

An acoustic matching agent 111 is disposed between the carriage 107 and the holding cup 119 that holds the object (breast 123) of a subject 121, Water is used as this acoustic matching agent 111.

A bed 117 that supports the subject 121 has an opening, through which the holding cup 119 holds the breast 123. A space between the breast 123 and the holding cup 119 is filled with an ultrasonic gel (not illustrated) for acoustic matching. The holding cup 119 is immovable.

The light emitted from the light source 101 propagates through the waveguide unit 103 that includes the articulated arm and radiated to the breast 123 through the radiating unit 105, the acoustic matching agent 111, and the holding cup 119. Then, a photoacoustic wave is generated in the breast 123. The generated photoacoustic wave is detected by the detection unit 109 and converted into electric signals.

The acquisition unit 165 generates a three-dimensional initial sound pressure distribution by using the UBP algorithm in the object from the electric signals collected by an electric signal collection unit 157. The three-dimensional initial sound pressure distribution is generated for each position of the radiating unit 105, that is, on an electric signal collecting position-by-electric signal collecting position basis. The three-dimensional light intensity distribution information in the object is generated by using the light diffusion equation in accordance with the information about the emitted-light-intensity distribution stored in the memory 301. This three-dimensional light intensity distribution information is generated for each position of the radiating unit 105. Furthermore, the optical absorption coefficient distribution in the object is acquired for each position of the radiating unit 105 by normalizing the initial sound pressure distribution by using the three-dimensional light intensity distribution information. By performing these steps over an entire scanning range of the radiating unit 105 and by superposing the initial sound pressure distributions acquired at the positions of the radiating unit 105 on one another and the optical absorption coefficient distributions acquired at the positions of the radiating unit 105 on one another, the three-dimensional initial sound pressure distribution and the three-dimensional optical absorption coefficient distribution of the entirety of the object are acquired.

The order of operations is not limited to the above-described order. Alternatively, the operations may be performed in the following order: that is, the three-dimensional initial sound pressure distribution of the entirety of the object is initially acquired; then, the three-dimensional light intensity distribution information of the entirety of the object is acquired; and from these, the three-dimensional optical absorption coefficient distribution of the entirety of the object is acquired.

A controller 151 includes a light source controller, a scanning controller, an electric signal collection controller, and a system controller, which controls the entirety of the information acquisition apparatus. The light source controller of the controller 151 controls the light source 101 so as to cause the pulsed light to he emitted at desired timing. According to the present embodiment, the light source 101 is controlled at a repetition frequency of 10 Hz.

The scanning controller of the controller 151 controls the scanning driver 153 so as to cause the radiating unit 105 to be desirably moved. Furthermore, the scanning controller issues an instruction to a scanning position acquisition unit 155 so as to cause the scanning position acquisition unit 155 to acquire the position information of the radiating unit 105 at the moment when the pulsed light is radiated to the object. According to the present embodiment, since the radiating unit 105 and the detection unit 109 are integrated with each other, the acquired position information of the radiating unit 105 also serves as the electric signal acquisition position information of the detection unit 109.

For example, upon reception of an instruction from the scanning controller, the scanning driver 153 causes the carriage 107, which supports the radiating unit 105 and the detection unit 109 integrated with each other, to perform spiral scanning. The light source controller issues an instruction to the light source 101 so as to cause the light source 101 to emit pulsed light 512 times at a repetition frequency of 10 Hz in accordance with the scanning. The number of positions of the radiating unit 105 is 512 at the moment when the pulsed light is radiated to the breast 123.

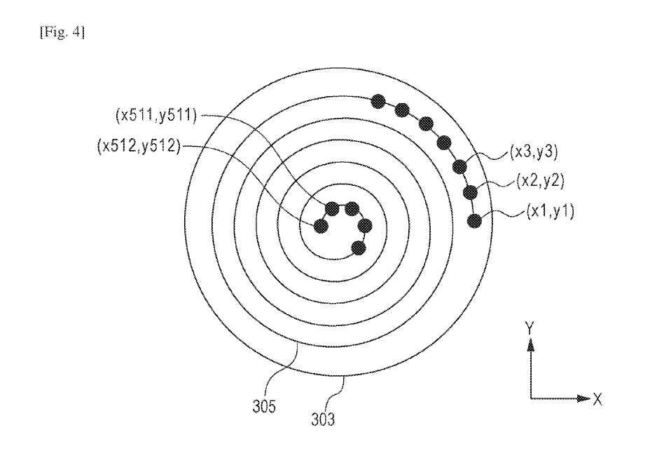

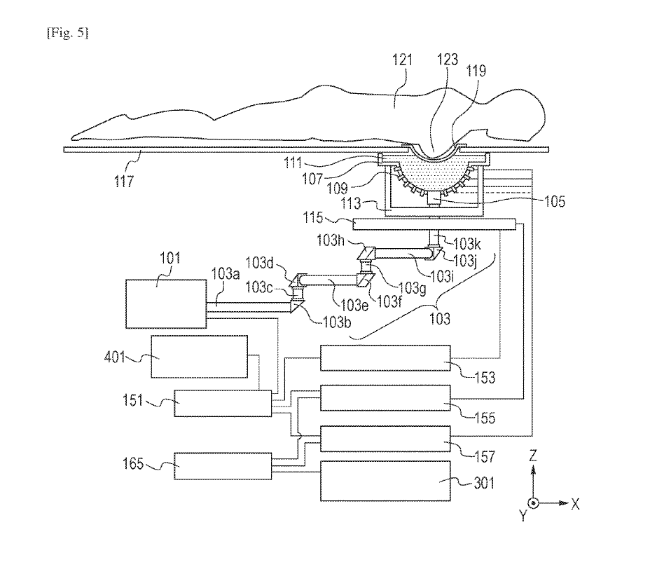

FIG. 4 illustrates scanning positions of the radiating unit 105 schematically illustrating a scanning path of the radiating unit 105 observed from the bed 117 side. Referring to FIG. 4, reference numeral 303 denotes a scannable range, and reference numeral 305 denotes a scanning path of the radiating unit 105. Black dots represent the scanning positions of the radiating unit 105 at the moment when the pulsed light is radiated to the breast 123, and (x1, y1) and the like represent the XY coordinates. By performing such scanning, the object information of the entire object can be acquired.

The electric signal collection controller of the controller 151 issues an instruction to the electric signal collection unit 157 so as to cause the electric signal collection unit 157 to collect signals having reached the detection unit 109. The signals are collected from 60 μs to 110 μs with the moment when the pulsed light is radiated to the object set as 0 μs. This duration of time corresponds to a distance of 75 mm in the object.

According to the present embodiment, the light is guided through the first waveguides in the same direction even in the waveguide unit that uses the articulated arm. Thus, variation of the emitted-light-intensity distribution relative to the scanning position of the radiating unit can be reduced. Accordingly, the information acquisition apparatus can be provided with which the light intensity distribution in the object can be accurately calculated. Furthermore, with this information acquisition apparatus, the object information can be accurately acquired.

The light source 101 according to the present embodiment is structured such that the emitted-light-intensity distribution of the light guided through the vertical waveguide 103c is asymmetric about the central axis of the vertical waveguide 103c. However, this is not limiting, The present invention is effective also in the following cases. That is, in the case where the center of the emitted-light-intensity distribution of the light guided through vertical waveguide 103c is misaligned with the central axis of the vertical waveguide 103c, in the case where the wave guiding direction of the light guided through the vertical waveguide 103c is not completely parallel to the direction of the central axis of the vertical waveguide 103c, and so force.

Furthermore, the radiating unit 105 may also serve as one of the first waveguide units (vertical waveguide 103k) that is closest to the object in the optical path through which the light emitted from the light source 101 is guided through the waveguide unit 103.

### Second Embodiment

FIG. 5 is a schematic view of an example of the information acquisition apparatus according to a second embodiment. The difference between the information acquisition apparatus according to the first embodiment and the information acquisition apparatus according to the present embodiment is that the information acquisition apparatus according to the present embodiment includes a region setting unit 401 that sets the region of interest of the object. Other than this feature, the information acquisition apparatus according to the present embodiment is the same as that of the first embodiment. In FIG. 5, the same elements as those illustrated in FIG. 1A are denoted by the same reference numerals and description thereof is omitted, The present embodiment is intended to be used in the case where the region of interest in the object is recognized in advance by, for example, palpation or an image acquired with another imaging apparatus using the method such as ultrasonography or magnetic resonance imaging (MRI).

The scanning controller of the controller 151 sets the scanning range and a scanning pattern of the radiating unit 105 in accordance with the region of interest set by the region setting unit 401 and controls the scanning driver 153 so as to cause the radiating unit 105 to be desirably moved.

The region setting unit 401 may set the region of interest in the object in accordance with a specification made by the operator on a monitor (not illustrated) or the region setting unit 401 may automatically set the region of interest.

The region of interest and the scanning range of the radiating unit 105 are described with reference to FIG. 6. Referring to FIG. 6, reference numeral 303 denotes a scannable range 303, and reference numeral 405 denotes a region of interest that has been set. Reference numeral 407 denotes the scanning path of the radiating unit 105 determined in accordance with the region of interest 405.

Where to set the region of interest depends on the object, and the number of positions where the region of interest can be set is infinite. Accordingly, the number of the scanning patterns of the radiating unit 105 that can be set is infinite. Even in such a case, since the radiated light intensity distribution of the light from the radiating unit 105 is substantially unchanged in accordance with the scanning position, the object information can be accurately calculated independently of where the region of interest 405 is set as is the case with the first embodiment.

### Third Embodiment

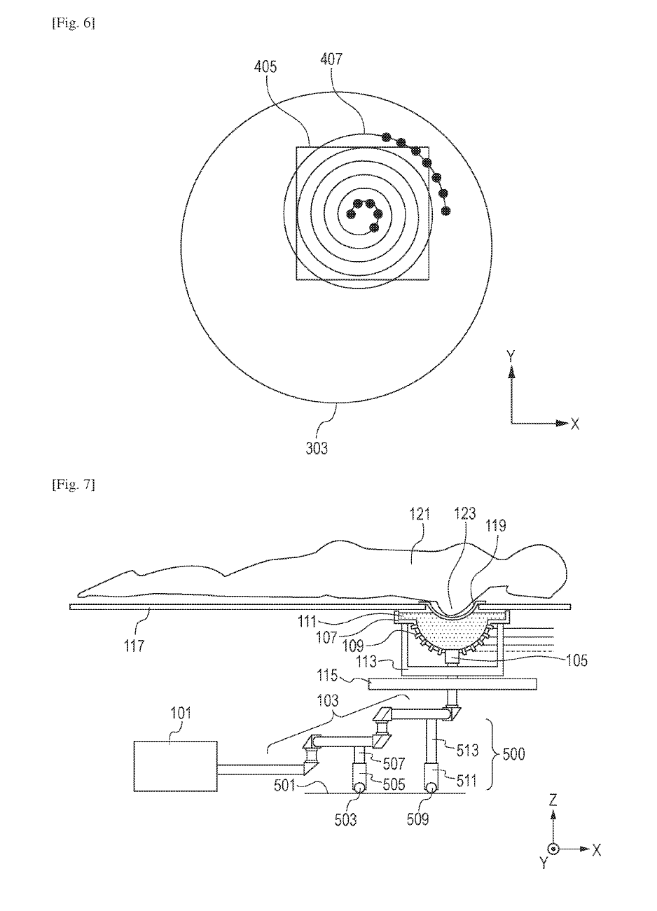

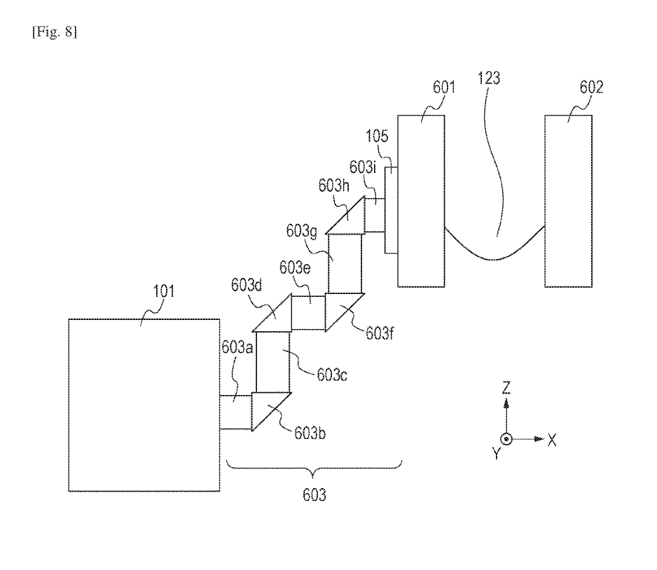

FIG. 7 is a schematic view of an example of the information acquisition apparatus according to a third embodiment. The difference between the information acquisition apparatus according to the first embodiment and the information acquisition apparatus according to the present embodiment is that the information acquisition apparatus according to the present embodiment includes a support unit 500 that supports the waveguide unit 103. Other than this feature, the information acquisition apparatus according to the present embodiment is the same as that of the first embodiment. The same elements as those illustrated in FIG. 1A are denoted by the same reference numerals and description thereof is omitted.

Specifically, the support unit 500 that supports the waveguide unit 103 includes balls 503 and 509 and ball receiving members 505 and 511. The balls 503 and 509 are disposed on a floor 501 so as to be movable along a surface of the floor 501. The ball receiving members 505 and 511 support the balls 503 and 509, respectively. Furthermore, the support unit 500 includes connecting members 507 and 513 that connect the ball receiving members 505 and 511 to the waveguide unit 103. The balls 503 and 509 allow the support unit 500 to be moved in a plane perpendicular the vertical direction. This support unit 500 reduces the likelihood of mechanical misalignment occurring when the radiating unit 105 performs the scanning, and accordingly, the radiating unit 105 can more reliably perform the scanning. Although it is not illustrated in FIG. 7, mechanisms that allow the lengths of the connecting member 507 and 513 to be adjusted are provided.

### Fourth Embodiment

Although examples of the optical scanning unit moved in the XY plane according to the first to third embodiments are described, the present invention is not limited to these. FIG. 8 is a schematic view of an example of a waveguide unit of the information acquisition apparatus according to a fourth embodiment. Other than this, the same structure as that of any one of the first to third embodiment can be applied. For example, the following structure according to the present embodiment may be used: the subject is laid on the bed in a prone position; the breast 123 is held by two holding plates 601 and 602; and a waveguide unit 603, which is an articulated arm, and the radiating unit 105 are moved in the YZ plane along the holding plates 601 and 602.

The waveguide unit 603 is an articulated arm that includes horizontal waveguides 603a, 603e, and 603i, vertical waveguides 603c and 603g, and articulations 603b, 603d, 603f, and 603h. According to the present embodiment, the radiating direction in which the light is radiated from the radiating unit 105 to the object (breast 123) is the +X direction. Thus, the first waveguides are the horizontal waveguides 603a, 603e, and 603i, and the second waveguides are the vertical waveguides 603c and 603g. According to the present embodiment, the light is guided through the horizontal waveguides 603a, 603e, and 603i in the same wave guiding direction, that is, in the +X direction.

With this structure, the radiated light intensity distribution of the light radiated from the radiating unit is not affected by the scanning position of the radiating unit 105. Accordingly, the same effects as those obtained with the first embodiment can be obtained.

Also according to the present embodiment, similarly to the first embodiment, in order to allow the radiating unit to scan in a two-dimensional manner, the information acquisition apparatus needs two or more first waveguides that are movable in the YZ plane. Furthermore, since an increase in the number of waveguides included in the waveguide unit 603 leads to a complex structure, the number of the first waveguides is preferably five or less.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions,

This application claims the benefit of Japanese Patent Application No. 2014-233799, filed Nov. 18, 2014, which is hereby incorporated by reference herein in its entirety.

1. An information acquisition apparatus comprising:
a light source;
a radiating unit that radiates to an object light emitted from the light source;
a waveguide unit that guides the light emitted from the light source to the radiating unit;
a detection unit that detects an acoustic wave generated by radiating the light from the radiating unit to the object and that outputs an electrical signal; and
an acquisition unit that acquires information of an inside of the object in accordance with the electric signal,
wherein the radiating unit is able to perform scanning,
wherein the radiating unit is connected to an end point of the waveguide unit,
wherein the waveguide unit includes
a plurality of first waveguides that guide the light in a direction parallel to a radiating direction in which the light is radiated from the radiating unit to the object,
at least one second waveguide that guides the light in an in-plane direction perpendicular to the radiating direction, and
a plurality of articulations that connect the plurality of first waveguides and the at least one second waveguide to one another and each include therein a mirror disposed so as to substantially perpendicularly bend a wave guiding direction of the light guided through the plurality of first waveguides and the at least one second waveguide,
wherein the plurality of first waveguides are connected to one side and another side of the at least one second waveguide with the plurality of articulations interposed therebetween, and
wherein the wave guiding direction of the light guided through one of the plurality of first waveguides located on the one side of the at least one second waveguide is identical to the wave guiding direction of the light guided through the other of the plurality of first waveguides located on the other side of the at least one second waveguide.

2. The information acquisition apparatus according to claim 1, further comprising:
a memory that stores in advance information about an emitted-light-intensity distribution of radiated light radiated from the radiating unit at one of a plurality of scanning positions that are scanned,
wherein the acquisition unit acquires the information of the inside of the object in accordance with the electric signal and the information about the emitted-light-intensity distribution stored in the memory.

3. The information acquisition apparatus according to claim 1, further comprising:
a support body that supports the radiating unit and the detection unit integrated with the radiating unit,
wherein the radiating unit and the detection unit are able to perform scanning in an integrated manner.

4. The information acquisition apparatus according to claim 1, further comprising:
a region setting unit that sets a region of interest of the object; and
a scanning controller that sets a scanning pattern and a scanning range of the radiating unit in accordance with the region of interest.

5. The information acquisition apparatus according to claim 1, further comprising:
a support unit that supports the waveguide unit,
wherein the support unit is movable in a plane perpendicular a vertical direction.

6. The information acquisition apparatus according to claim 1,
wherein the at least one second waveguide includes two or more second waveguides that are movable in the in-plane direction.

7. The information acquisition apparatus according to claim 1,
wherein the radiating unit also serves as one of the plurality of first waveguides disposed closest to the object out of the plurality of first waveguides in an optical path in which the light emitted from the light source is guided through the waveguide unit.

8. The information acquisition apparatus according to claim 1,
wherein the light guided through one of the plurality of first waveguides closest to the light source out of the plurality of first waveguides in an optical path in which the light emitted from the light source is guided through the waveguide unit has an emitted-light-intensity distribution that is asymmetric about a central axis of the one of the plurality of first waveguides.
